(19) United States
(12) Patent Application Publication
(10) Pub. No.: US 2010/0049136 A1
(43) Pub. Date: Feb. 25, 2010

(54) Guide Wire and Method of Use Thereof

(75) Inventor: Frank J. Fischer, JR., Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/193,856

(22) Filed: Aug. 19, 2008

(51) Int. Cl. A61M 25/09

(52) U.S. Cl. 604/164.13

(57) ABSTRACT

The present invention generally relates to a medical device and specifically to a guide wire for reversing the direction of catheterization within a body lumen and to a method of use thereof. In one embodiment, the guide wire includes a first elongated member having a second elongated member attached to the distal end. A spring element joins the distal end of the second elongated member to a third elongated member. The third elongated member is movable between a first position folded upon the second elongated member and a second position extending distally from the second elongated member.

### TECHNICAL FIELD

The present invention generally relates to a guide wire allowing for reversal of direction of catheterization of a body lumen and to a method of use thereof.

### BACKGROUND

Numerous medical procedures involve the introduction of a catheter or a similar device into a lumen in the body. Structures which can be accessed by catheterization include the blood vessels, the bowel and digestive tract, the bile ducts, the urinary tract and several others. Known catheterization procedures include the positioning and inflation of balloons for opening a constricted vessel, the positioning and expansion of stents for maintaining an open lumen in a constricted vessel, the intravenous administration of nutrient fluids, the delivery of whole blood or blood products, the sampling of blood and the administration of chemotherapeutic agents or other drugs.

The first step in the performance of such procedures is the establishment of a site through the skin by which access is had to the lumen. A guide wire is introduced into the lumen and the catheter advanced over the guide wire into the lumen in one direction or another to a desired location. If this direction is opposite to the direction of fluid flow in the vessel or other structure, it is referred to as the “retrograde” direction. On the other hand, if this direction is the same as the direction of fluid flow in the vessel, it is referred to as the “antegrade” direction. By way of example, catheterization of the femoral artery, axillary artery or brachial artery for access to the vessels of the abdomen or thorax is carried out by advancement of the catheter in the retrograde direction.

It is sometimes the case that a lumen cannot be catheterized in the required direction. For example, accessing the superficial femoral artery of critical limb ischemia patients with an ipsi-lateral approach is difficult if the patients are morbidly obese. Accessing using a needle angling downward is difficult because of the obesity. In such cases, it is advantageous to access with the needle upward and deliver the guide wire in this configuration. However, it has generally been found to be very difficult, sometimes even impossible, to reverse the direction of catheter advancement through the initial access site.

It should thus be clear that it would be highly desirable to have an apparatus for reversing the direction of catheterization within a patient, either retrograde to antegrade, or antegrade to retrograde. It would also be highly desirable to have such an apparatus that is relatively simple and reliable in construction and use, and which is relatively low in cost, at least in comparison to the costs and risks incurred in establishing a second access site.

### BRIEF SUMMARY

One aspect provides a guide wire including a first elongated member having a proximal end and a distal end. A second elongated member is attached to the distal end of the first elongated member and to a spring element joining the distal end of the second elongated member to a third elongated member. The third elongated member is movable about the spring element between a first position folded upon the second elongated member and a second position extending distally from the second elongated member.

In one embodiment, the second elongated member has a maximum cross sectional dimension that is less than a maximum cross sectional dimension of the first elongated member. In another embodiment, the second elongated member and the third elongated member each have a maximum cross sectional dimension that is equal to or less than a maximum cross sectional dimension of the first elongated member.

In yet another embodiment, the guide wire comprises a self-expanding nickel titanium alloy. In another embodiment, the third elongated member is spring-biased to extend distally from the second elongated member.

In one embodiment, the maximum cross sectional dimension of the first elongated member is between 0.04 mm and 0.02 mm. In yet another embodiment, the maximum cross sectional dimension of the second elongated member is between 0.02 mm and 0.01 mm.

Another aspect provides a method of reversing direction of catheterization of a guide wire into a body lumen of a patient. The method includes inserting a needle into the lumen in a first direction and inserting the distal end of a guide wire of the present embodiments into the needle and into the lumen. During insertion, the third elongated member is folded upon the second elongated member.

The needle is removed and, in certain embodiments, an inducer sheath is advanced over the guide wire until the inducer sheath is positioned over the first elongated member. The first elongated member is rotated whereby the third elongated member is moved about the spring element to a position extending distally from the second elongated member.

### BRIEF DESCRIPTION OF THE DRAWINGS

FIGS. 1(a)-1(b) are illustrations of one embodiment of a guide wire. FIG. 1(a) illustrates the guide wire is a folded configuration. FIG. 1(b) illustrates the guide wire in an expanded configuration.

FIGS. 2(a)-(c) are illustrations of three embodiments of a guide wire.

FIG. 3 is an illustration by one embodiment of a guide wire.

FIGS. 4(a)-(e) are schematic illustrations of a method of reversing the direction of a guide wire within a body lumen.

### DETAILED DESCRIPTION

As used herein, the term “proximal” refers to that portion of the device closest to a physician when placing the device in the patient lumen, and the term “distal” refers to that portion of the device closest to the end inserted into the patient's body lumen. The terms “antegrade” and “retrograde” refer to directions relative to the direction of fluid flow within the body lumen. The antegrade refers to the direction of fluid flow within the body lumen.

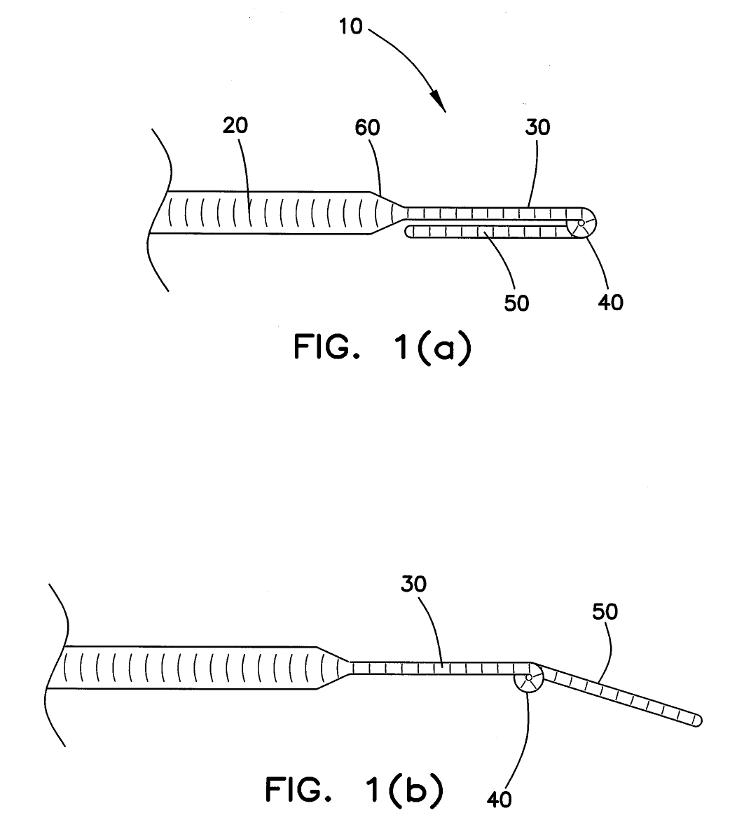

Referring now to FIG. 1(a), this figure illustrates a guide wire 10 including first elongated member 20. Second elongated member 30 extends distally from the distal end of first elongated member 20. Spring element 40 connects the distal end of second elongated member 30 to third elongated member 50. Third elongated member 50 is movable about spring element 40 between a position where it is folded against second elongated member 30 and a position where it extends distally from second elongated member 30. FIG. 1(a) illustrates a configuration in which third elongated member 50 is folded against second elongated member 30. FIG. 1(b) illustrates a configuration in which third elongated member 50 extends distally from spring element 40 and from second elongated member 30.

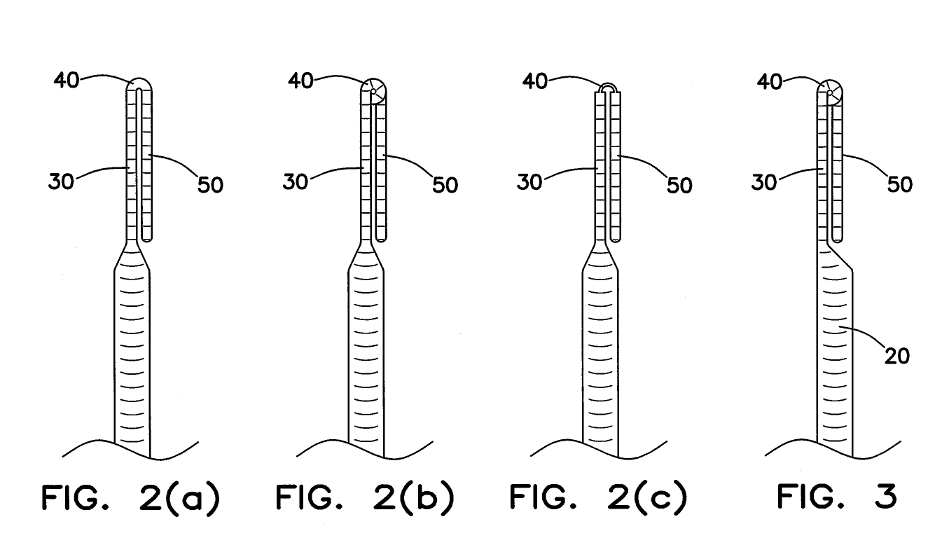

FIGS. 2(a)-(c) illustrate alternative embodiments of spring element 40. FIG. 2(a) illustrates an embodiment in which spring element 40 is a simple bend between second elongated member 30 and third elongated member 50. FIG. 2(b) illustrates an embodiment in which spring element 40 is a coil having approximately one and a half turns when second elongated member 30 is folded against third elongated member 50. FIG. 2(c) illustrates an embodiment in which spring element 40 is a simple bend between second elongated member 30 and third elongated member 50, where the cross sectional dimension of spring element 40 is less than that of second elongated member 30 or third elongated member 50.

Second elongated member 30 may be an extension of first elongated member 20 formed by reducing the cross section of a portion of first elongated member 20. Such an embodiment is illustrated in FIG. 1(a). Here, the cross sectional dimension of guide wire 10 is reduced at taper 60 between first elongated member 20 and second elongated member 30. Of course, the present embodiments also include those not having a taper, resulting in an abrupt change in cross section between first elongated member 20 and second elongated member 30.

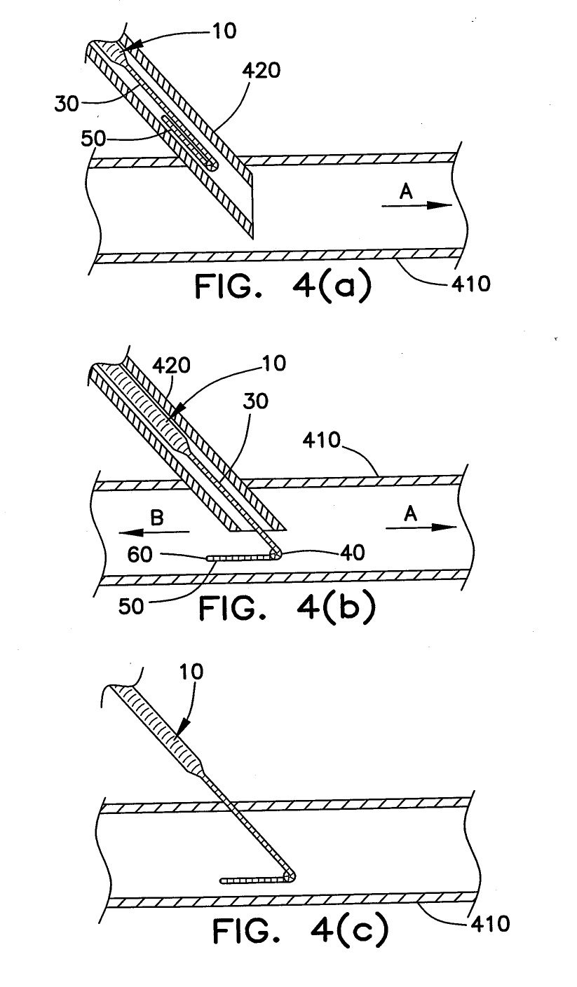

FIG. 3 illustrates an embodiment where one side of second elongated member 30 is continuous with one side of first elongated member 20. Third elongated member 50 is again folded at spring element 40. Here the maximum cross-sectional dimension of second elongated member 30 and third elongated member 50 are approximately half that of first elongated member 20. This configuration, allows second elongated member 30 and folded third elongated member 50 to be accommodated within a region equivalent to an extension of the cross sectional dimension of first elongated member 20. Other embodiments include those in which second elongated member 30 is offset to one side of first elongated member 20. Reducing the cross sectional dimension of the guide wire is advantageous in that it allows the guide wire to be introduced into a body lumen through a smaller gauge needle.

Guide wire 10 or portions thereof may be formed of materials such as stainless steel, tantalum, nickel-titanium alloys, such a NITINOL®, gold, silver, tungsten, platinum, cobalt-chromium alloys and iridium, all of which are commercially available metals or alloys. Spring element 40 may be formed of similar materials. In one embodiment, spring element 40 is formed from a self-expanding nickel-titanium alloy, such a NITINOL®.

In one embodiment, the maximum cross sectional dimension of first elongated member 20 is such that guide wire 10 may be introduced into a body lumen through a hypodermic needle, for example, an 18 gauge hypodermic needle. In one embodiment, the maximum cross sectional dimension of first elongated member 20 is between 0.04 mm and 0.02 mm. In another embodiment, the maximum cross sectional dimension of first elongated member 20 is between 0.035 mm and 0.025 mm.

In other embodiments, the maximum cross sectional dimension of second elongated member 30 is less than the maximum cross sectional dimension of first elongated member 20. In one embodiment, the maximum cross sectional dimension of second elongated member 30 is half or less than half the maximum cross sectional dimension of first elongated member 20. In another embodiment, the maximum cross sectional dimension of second elongated member 30 is between 0.02 mm and 0.01 mm. In yet another embodiment, the maximum cross sectional dimension of second elongated member 30 is between 0.017 mm and 0.013 mm.

In another embodiment, the maximum cross sectional dimension of third elongated member 50 is approximately the same as that of second elongated member 30. In yet another embodiment, the maximum cross sectional dimension of second elongated member 30 and the maximum cross sectional dimension of third elongated member 50 are both half or less than half the maximum cross sectional dimension of first elongated member 20.

In another embodiment, the combined maximum cross sectional dimension of the second elongated member 30 and the third elongated member third elongated member 50 is equal to or less than the maximum cross sectional dimension of first elongated member 20.

The lengths of second elongated member 30 and third elongated member 50 are chosen to allow guide wire 10 to be introduced into the body lumen such that the end of third elongated member 50 attached to spring element 40 is delivered beyond the distal end of the delivery needle and is hence free to move away from a folded position against second elongated member 30. In one embodiment, second elongated member 20 is between 3.5 cm and 4.5 cm in length. In another embodiment, second elongated member 20 is approximately 4.1 cm in length. In yet another embodiment, third elongated member 50 is between 2.5 cm and 3.5 cm in length. In another embodiment, third elongated member is approximately 3.0 cm in length.

In one embodiment, third elongated member 50 is spring-biased at spring element 40 so that second elongated member 30 extends away from second elongated member 30 when free to do so, for example, then third elongated member 50 is inserted into a body lumen beyond the distal end of a delivery needle. In such embodiments, the spring strength or stiffness of spring element 40 is chosen so that although third elongated member 50 moves away from second elongated member 30 when not constrained, it does not do so with sufficient force to damage the wall of the body lumen. Selection of appropriate spring strength may be achieved to methods known to those skilled in the art. In one embodiment, the end of third elongated member 50 that is not attached at spring element 40 is blunt and/or includes a cap to prevent or reduce damage to the wall of the body lumen.

The guide wire may have typical guide wire dimensions. The guide wire length may generally be about 90 to about 300 cm, and for use within a patient's coronary system available guide wires are typically about 180 cm in length.

In one embodiment, the body lumen is a vascular vessel. For example, the device may be used to reverse direction of catheterization of the femoral artery. Accessing the superficial femoral artery of critical limb ischemia patients with an ipsi-lateral approach is difficult if the patients are morbidly obese. Accessing using a needle angling downward is difficult because of the obesity. In such cases, it is advantageous to access with the needle upward and deliver the guide wire in this configuration. In other embodiments, the body lumen is, for example, the bowel and digestive tract, the bile ducts or the urinary tract. However, the devices and methods of the present embodiments may be used to reverse the direction of the guide wire within other body lumens.

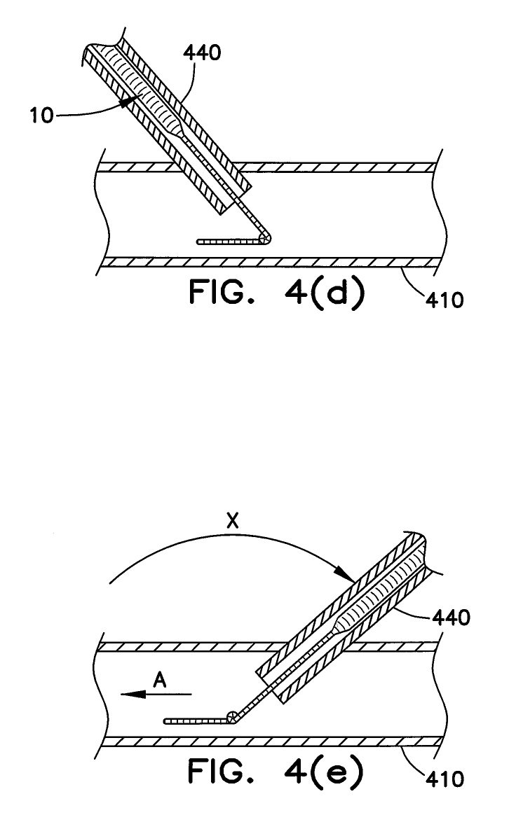

FIGS. 4(a)-(e) illustrate a procedure for reversing the direction of the guide wire within a body lumen, for example, a vascular vessel. FIG. 4(a) shows vessel 410 with needle 420 inserted in a first direction “A.” Guide wire 10 is inserted into needle 420. During delivery through needle 420, third elongated member 50 is positioned against second elongated member 30. In FIG. 4(b), guide wire 10 is inserted into vessel 410 so that end 60 of third elongated member 50 is moved beyond the distal end of needle 420. Third elongated member 50 moves away from second elongated member 30 as spring member 40 straightens. End 60 of third elongated member 50 points is direction “B” although needle 420 and second elongated member 30 point towards direction “A.”

Needle 420 is removed leaving guide wire 10 in place in vessel 410, as is illustrated in FIG. 4(c). FIG. 4(d) shows inducer sheath 440 placed over guide wire 10 and into vessel 410 until the distal end of inducer sheath 440 is positioned near the proximal end of the spring element. In FIG. 4(e), inducer sheath 440 is rotated in direction “X” to reverse the direction of guide wire 10, so that guide wire 10 now points in the direction of arrow “A.” In other embodiments, inducer sheath is replaced by a dilator or another device providing sufficient stiffness to allow rotation of guide wire 10.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A guide wire comprising:
a first elongated member having a proximal end and a distal end;
a second elongated member having a proximal end attached to the distal end of the first elongated member;
a spring element joining a distal end of the second elongated member to a first end of a third elongated member, wherein the third elongated member is movable about the spring element between a first position folded upon the second elongated member and a second position extending distally from the second elongated member.

2. The guide wire of claim 1, wherein the second elongated member has a maximum cross sectional dimension that is less than a maximum cross sectional dimension of the first elongated member.

3. The guide wire of claim 1, wherein the second elongated member and the third elongated member each have a maximum cross sectional dimension that is less than a maximum cross sectional dimension of the first elongated member.

4. The guide wire of claim 3, wherein the second elongated member and the third elongated member each have a maximum cross sectional dimension that is at the most half the maximum cross sectional dimension of the first elongated member.

5. The guide wire of claim 4, wherein the second elongated member and the folded third elongated member are accommodated within a region equivalent to an extension of the maximum cross sectional dimension of the first elongated member.

6. The guide wire of claim 1, wherein the guide wire comprises a material selected from the group consisting of stainless steel, tantalum, nickel-titanium alloys, gold, silver, tungsten, platinum, cobalt-chromium alloys and iridium.

7. The guide wire of claim 1, wherein the guide wire comprises a self-expanding nickel titanium alloy.

8. The guide wire of claim 1, wherein the third elongated member is spring-biased at the spring element to extend distally from the second elongated member.

9. The guide wire of claim 2, wherein the maximum cross sectional dimension of the first elongated member is between 0.04 mm and 0.02 mm.

10. The guide wire of claim 9, therein the maximum cross sectional dimension of the second elongated member is between 0.02 mm and 0.01 mm.

11. The guide wire of claim 1, wherein the spring element comprises a self-expanding nickel titanium alloy.

12. The guide wire of claim 1, wherein, then the third elongated member is in the first position folded upon the second elongated member, the guide wire has a maximum cross sectional dimension allowing for insertion through a 18 gauge hypodermic needle.

13. A method of reversing direction of insertion of a guide wire into a body lumen of a patient, the method comprising:
providing a guide wire comprising:
a first elongated member having a proximal end and a distal end;
a second elongated member having a proximal end attached to the distal end of the first elongated member; and
a spring element joining the distal end of the second elongated member to a first end of a third elongated member, wherein the third elongated member is movable about the spring element between a first position folded upon the second elongated member and a second position extending distally from the second elongated member and wherein the third elongated member is folded upon the second elongated member;
inserting a needle into the body lumen in a first direction;
inserting a distal end of the guide wire into the needle and into the vessel;
removing the needle from the body lumen; and
rotating the first elongated member whereby the third elongated member is moved about the spring element to a position extending distally from the second elongated member.

14. The method of claim 13, further comprising advancing an inducer sheath over the first elongated member before rotating the first elongated member.

15. The method of claim 13, wherein the body lumen is a femoral artery.

16. The method of claim 13, wherein the guide wire comprises a material selected from the group consisting of stainless steel, tantalum, nickel-titanium alloys, gold, silver, tungsten, platinum, cobalt-chromium alloys and iridium.

17. The method of claim 13, wherein the third elongated member is spring-biased at the spring element to extend distally from the second elongated member.

18. The method of claim 13, wherein the second elongated member and the folded third elongated member are accommodated within a region equivalent to an extension of the maximum cross sectional dimension of the first elongated member.

19. The method of claim 13, wherein the needle is an 18 gauge hypodermic needle.
